(19) United States
(12) Patent Application Publication
(10) Pub. No.: US 2006/0037085 A1
(43) Pub. Date: Feb. 16, 2006

(54) Animal model for fetal alcohol syndrome and methods of treatment

(76) Inventors: Ying Peng, Guangzhou (CN); Marie Chia-Mi Lin, Ap Lei Chau (HK); Hsiang-Fu Kung, Hong Kong (HK); Pai-Hao Yang, Shek Tong Tsui (HK)

Correspondence Address: COOPER & DUNHAM, LLP, 1185 AVENUE OF THE AMERICAS, NEW YORK, NY, 10036, US

(21) Appl. No.: 11/192,194

(22) Filed: Jul. 28, 2005

Related U.S. Application Data
(60) Provisional application No. 60/601,443, filed on Aug. 13, 2004.

(51) Int. Cl. A01K 67/027

(52) U.S. Cl. 800/3

(57) ABSTRACT

A Xenopus laevis (the African clawed frog) embryo model is provided to study the effects of alcohol on fetal development. Exposure of Xenopus embryos in specific developmental stages to alcohol results in tadpoles with microencephaly and growth retardation, in a dose- and time-dependent manner, similar to those observed in human fetal alcohol syndrome (FAS). The invention further provides methods for screening an agent to determine its usefulness for preventing or treating FAS. Moreover, the invention provides methods for preventing or treating FAS in an animal by administering an agent, such an agent includes vitamin C and a catalase, which causes or enhances an expression of Pax6 that is a neural and eye marker. In addition, the invention provides methods for preventing or treating FAS by administering an agent, such as vitamin C, which causes suppression of NF-κB activation.

This application claims priority of provisional application U.S. Ser. No. 60/601,443, filed Aug. 13, 2004, the contents of which is being incorporated herein by reference.

### FIELD OF THE INVENTION

This invention relates to an animal model for fetal alcohol syndrome, a critical target in alcohol-induced fetal microcephaly and other birth defects associated with fetal alcohol syndrome, methods of treating fetal alcohol syndrome, and methods for screening compounds which can mitigate the birth defects associated with fetal alcohol syndrome.

### BACKGROUND OF THE INVENTION

Fetal alcohol syndrome (FAS), which is associated with maternal alcohol abuse during pregnancy, represents a major public health issue. FAS is characterized by abnormalities in eye/gut development, the central nervous system (CNS), along with a smaller brain, growth retardation, and characteristic facial dysmorphology in the fetus of mothers who abuse alcohol during pregnancy. The incidence of FAS is 0.5-3 in 1000 births in the United States. Defects in the development of the CNS, which usually lead to mental retardation and abnormally small head, i.e., microencephaly (Peng, Neurobiology of Disease, 2004), are among the most significant effects of prenatal ethanol exposure. Other defects often observed with FAS are eye defect (Peng, IOVS, 2004) and gut defect (Peng, J. Mol. Biol., 2004).

Ethanol exerts profound and divergent effects on the developing brain. Ethanol induces widespread apoptotic neurodegeneration in the developing rat forebrain, disrupts the development of astroglial cells and interferes with the function of GABA, glutamatergic, serotonergic, dopaminergic, cholinergic, and opioid neuronal systems. Oxidative damage is one of many proposed mechanisms thought to contribute to nervous system dysfunction characterized in FAS. Rat, mouse and chicken embryo model systems have been used to characterize the nature of alcohol-induced brain damage. Although the effects of ethanol exposure on the developing brain are well documented, the mechanisms by which ethanol leads to these effects are not clearly understood, owing in part to the complexity of the model systems currently in use. (Also, see K. Stratton. (eds), 1996; C. Ikonomidou, 2000; C. Guerri, 1997; M. Guizzetti, 2000; L. G. Costa, 1999; M. J. Eckardt, 1998; H. C. Becker, 1996; C. E. Chmielewski, 1997; B. Bupp, 1998).

In view of the wide use of alcohol in our society, a need for a comprehensive and systematic study is warranted. In order to thoroughly investigate and test all potential mechanisms and valuable neuroprotective agents, a simpler model for the action of alcohol on developing embryos is required. Such a model provides a mechanism for testing agents to determine whether they are useful for the prevention or treatment of FAS.

### SUMMARY OF THE INVENTION

The present invention provides a Xenopus laevis (African clawed frog) model system to study the effect of alcohol on brain development and growth retardation. The dosage and minimum duration of treatment with ethanol that is required for an obvious effect can be determined with the system of the present invention. Using this model, protective agents against alcohol-induced injuries can also be determined. For example, vitamin C was found to effectively prevent alcohol-induced ROS production and NF-κB activation in parallel to the protection against growth retardation and microencephaly.

Xenopus laevis (Xenopus) is a gentle, freshwater animal that can be induced by a hormone, such as human chorionic gonadotropin (HCG) injection, to lay eggs repeatedly. These features, coupled with the large size of the embryos, which allows micromanipulation and microinjection. In addition, their rapid rate of development makes Xenopus an excellent animal model for analyzing early vertebrate development.

Moreover, Xenopus laevis is one of the simplest vertebrates having well-characterized developmental stages. A large number of embryos can be obtained at one time and the effects of alcohol on embryonic development can be studied in detail. In addition, Xenopus embryos can be developed outside their mothers and hence their incubation environments can be controlled precisely. With well-documented developmental processes, the effect of alcohol on the embryos can be easily assessed; it also allows rapid assessment of early developmental processes. The metabolic effects of intraperitoneal injection of ethanol on the maternal system on embryo development can be easily removed.

The present invention provides Xenopus as an animal model of fetal alcohol syndrome. For example, exposure of Xenopus embryo models of the present invention to ethanol between initial neural plate stage to late neurula stage was shown to be required and sufficient to produce eye defects, delayed gut development, and small head/body phenotypes similar to those phenotypes observed in human and mouse FAS. This result is consistent with a previous report, which shows that exposure of Xenopus embryos to ethanol from early gastrulation stage to the late neurula stage produced tadpoles with significantly smaller heads. The ethanol effect is readily reversible and does not produce a significant effect on tadpoles when ethanol is either removed prior to the mid-neurula stage or administered after late-neurula stage. These results suggest that the changes acquired during the initial to late neurula stage are the critical, and probably proximal causes of FAS; and that there is a window of opportunity for intervention. The use of model of the present invention therefore narrowed the window of alcohol exposure required. This observation in this Xenopus model is also consistent with the published data in mouse and chicken models of FAS.

The Xenopus model of the present invention can also be used to demonstrate that an antioxidant, ascorbic acid (vitamin C), inhibited alcohol-induced reactive oxygen species (ROS) production and NF-κB activation, and protected embryos against alcohol induced microencephaly as well as growth retardation. These results suggest the involvement of NF-κB and oxidative stress in alcohol-mediated developmental defects, and indicate the potential use of vitamin C as a new and effective protective agent against FAS.

In addition, using the Xenopus model of the present invention, it was found that the expression of several key neural genes was reduced. These genes include xPax6, xOtx2, xSox3, xSox2, and xNCAM, of which Pax6 was the most vulnerable. Microinjection of Pax6 into the Xenopus model dose-dependently rescued alcohol-induced microcephaly and restored the expression of xOtx2, xSox3, xSox2, and xNCAM. To test whether reactive oxygen species (ROS) is the upstream signal for alcohol-induced microcephaly and xPax6 suppression, catalase was overexpressed in Xenopus embryos. Catalase not only decreased alcohol-induced H₂O₂ formation, but also fully restored Pax6 expression and reversed microcephaly. In contrast, xPax6 and catalase could only provide partial protection against growth retardation. These results illustrate for the first time the critical role of H₂O₂-mediated Pax6 suppression in alcohol-induced microcephaly and suggest the presence of additional mechanisms or treatments for alcohol-induced fetal growth retardation.

### BRIEF DESCRIPTION OF THE DRAWINGS

The above and other features and advantages of the invention will be more readily understood from the following detailed description which is provided in connection with the accompanying drawings, in which:

FIGS. 1A through 1C shows the establishment of alcohol treatment conditions to produce Xenopus embryos with phenotypes similar to those observed in human FAS. Treated embryos were examined when the control embryos grown in the absence of ethanol reached tadpole stage.

FIG. 1A shows that ethanol dose-dependently affects the development of Xenopus embryo neural structures. The embryos were cultured in 30% MMR (Modified Marc's Ringer; with 0.5%, 0.6% and 1% ethanol and without removal of the ethanol until late neurula stage (Stage 22).

FIG. 1B shows photos of tadpoles exposed to ethanol at stages 13, 16, and 20, which appear to produce severe defects compared to the earlier or later exposure. Embryos were initially exposed to 0.6% ethanol at stages 9, 11, 13, 16, 18, 20, 23, 25 and 40 without removal of the ethanol until observation at stage 45.

FIG. 1C shows the results of ethanol treatment of tadpoles for 12 hours. To determine the minimum duration required to produce fetal deformations, embryos at stage 13 were exposed to 0.6% of ethanol for 1, 2, 4, 6, 12, 24, 36 and 48 hours. The embryos were then transferred to fresh MMR medium and allowed to grow until the control group reached tadpole stage. A 12-hour exposure (from initial neural plate stage to late neurula, stages 13-22) consistently produced an increased mortality and marked reduction in head and body sizes compared to the control group. The left panel: the control group; the middle panel: the 12-hour ethanol treated group; and the right panel: the group pretreated with 100 μM ascorbic acid for 2 hours before the commencement of a 12-hour ethanol treatment.

FIGS. 2A through 2M show histological analysis of the coronal brain sections of the tadpoles showing the effect of alcohol, and vitamin C on alcohol-induced injuries. Representative sections for the forebrain (FIGS. 2A through 2C), diencephalon (FIGS. 2D through 2I) and tectum areas (FIGS. 2J through 2L) were shown. FIG. 2M shows vitamin C upregulated NCAM expression in ethanol treated embryos. RT-PCR analysis of NCAM and Pax6 expression in embryos treated by indicated conditions. EF-1α expression was used as an internal control for equal RNA loading.

FIG. 3 shows representative photos of deformed tadpoles at stage 40 which had been exposed to varied concentrations (0.3%, 0.6% and 1%) of alcohol for 12 hours (from stage 13 to stage 22). The photos demonstrate alcohol-induced microcephaly in Xenopus embryos. Upper panel is a lateral view, middle panel is a dorsal view, and lower panel shows the heads of 0%, 0.3%, 0.6% and 1% alcohol-treated tadpoles, respectively. The values at the bottom indicate the frequencies of embryos exhibiting microcephaly and growth retardation.

FIG. 4 shows the downregulation of various neural gene expressions by alcohol treatment. (A) RT-PCR analysis of molecular markers which was performed using animal caps treated with various concentrations of alcohol, from 0.1% to 1% as indicated. Pax6: a neural and eye marker; NCAM: a pan-neural marker; Otx2: an anterior neural region marker; Sox2: a pan-neural marker; Sox3: a prospective neuroectoderm marker; and Vent1: a general ventral marker. The expression of elongation factor, EF-1α, was used as an internal control for equal RNA loading. (B) A graph showing the relative mRNA levels of the neural markers at increasing concentrations of alcohol.

FIG. 5 shows photos of embryos that are rescued from microcephaly by xPax6 expression in a dose-dependent manner. Embryos at 2-cell stage were injected with pCS2+ or varied doses of pCS2+xPax6 plasmid as indicated, and then treated with 0.6% alcohol for 12 hours from stage 13 to stage 22. Upper panel is a lateral view and lower panel is the dorsal view. The values indicated at the bottom of the figures represent the average values of body length (Length) and head size (Head) calculated as a percentage of the control non-alcohol-treated group.

FIG. 6 shows RT-PCR analysis of the effects of xPax6 expression on the expression of down stream neural genes. RT-PCR analysis was performed using animal caps microinjected with 100 pg of either pCS2+ or pCS2+xPax6 expression plasmid and then treated with 0.6% alcohol.

FIG. 7 shows that overexpression of human catalase significantly decreased alcohol-induced production of ROS and restored the expression of Pax6 and other neural genes. In each experiment, embryos were microinjected with 100 pg of either pCMS or pCMS-CAT expression plasmid at 2-cell stage, and treated with 0.6% alcohol from stage 13 until stage 22. At stage 22, embryos were harvested and analyzed. (A) Distribution of the injected pCMS-CAT plasmid as indicated by the EGFP (enhanced green fluorescent protein) fluorescence. Left panel photo was taken under light microscope and right panel under fluorescence microscope; (B) Western immunoblotting analysis of the expression of catalase protein. Lanes 1 and 3, injected with pCMS; lanes 2 and 4, injected with 50 and 100 pg of pCMS-CAT respectively. Actin immunoblotting is shown as a loading control. (C) Effects of catalase on alcohol-induced H₂O₂ production. The asterisk denotes a significant difference (p<0.05) from the pCMS vector-injected group; (D) RT-PCR analysis of gene expression in the animal cap assay.

FIG. 8 shows the effects of vitamin C on alcohol-induced ROS production and NF-κB activation. (A) Embryos were pretreated with or without vitamin C two hours before alcohol treatment (0.6%, 12 h, from stage 13 to 22). The embryos were harvested at stage 22 and the hydrogen peroxide level measured by the colorimetric quantitation of hydrogen peroxide method (total hydroperoxides), according to the protocols provided by the manufacturer (BIOXYTECH H202-560 kit, OXIS International, Inc., USA); (B) The NF-κB mediated transcriptional activity was measured using NF-κB luciferase reporter plasmid (pLuc-NF-κB) containing IL-6 promoter region with four repeats of NF-κB binding sites.

FIG. 9 shows that alcohol induces growth retardation and delayed gut development in Xenopus embryos. Midneurala stage embryos were kept in either the control 10% MSS medium or the same medium supplemented with 0.5% alcohol. The embryos were allowed to grow until the control embryos (no alcohol) reached the tadpole stage (stages 45-46). All the embryos were then fixed and their body length and gut morphologies were recorded. (A) The lateral views of the control (left) and alcohol-treated embryos (right). (B) Representative pictures showing the gut morphologies of the control (left panel) and 0.5% alcohol-treated embryos (right panels). Note that when the control embryos reached stages 45-46, the alcohol-treated embryos were still at stages 41-44 as indicated. (C) When compared to the control embryo (left), the sagittal section of an alcohol-treated embryo (right) showed a retarded development of the intestine, as demonstrated by a small number of intestinal loops and abnormally large gut lumen. Lower panels represent high power magnification of the boxed areas.

FIG. 10 shows that histological analyses of the stage 23 control (left panels) and 0.5% alcohol-treated embryos (right panels). Top: Sagittal sections of the control and alcohol-treated embryos were stained with H&E. The boxes indicate specific regions of the gut and the head mesenchyme. Bottom: High power magnification of the boxed areas showing the magnified views of the foregut, midgut, hindgut and head mesenchyme. Abbreviations: FG, foregut; HG, hindgut; HM, head mesenchyme; MG, midgut.

FIG. 11 shows (A) Correlations between delayed gut development and fetal growth. Embryos were injected with pCMS, pCMS-mtPRDX5, pCMS-cpPRDX5 or pCMS-Catalase expression plasmid, and then maintained in medium with or without 0.5% alcohol. When the tadpoles of control group reached stages 45-46, all the tadpoles were fixed and their gut developmental stages and body lengths were measured and used for the correlation studies. (B) Expression of PRDX5 and catalase in embryos differentially restore gut markers expression. Embryos were injected with pCMS, pCMS-mtPRDX5, or pCMS-Catalase expression plasmid, and then treated with or without alcohol as indicated. The expression of the gut markers was determined by RT-PCR. (C) Schematic presentation of the molecular basis of alcohol-induced delay in gut development.

### DETAILED DESCRIPTION OF THE INVENTION

In the following detailed description, reference is made to the accompanying drawings, which form a part hereof and show by way of illustration specific exemplary embodiments in which the invention may be practiced. These embodiments are described in sufficient detail to enable those skilled in the art to practice the invention, and it is to be understood that other embodiments may be utilized, and that changes may be made without departing from the spirit and scope of the present invention. The progression of processing steps described is exemplary of embodiments of the invention; however, the sequence of steps is not limited to that set forth herein and may be changed as is known in the art, with the exception of steps necessarily occurring in a certain order.

Embryo collection and experimental conditions for Xenopus model. Xenopus laevis embryos were obtained by in vitro fertilization as previously described (Peng, 2000). Developmental stages of embryos were determined using criteria of Nieuwkoop and Faber (Nieuwkoop and Faber, 1994). In each experiment, embryos at the 2-4 cell stage were collected from the dejellied embryos. After reaching the indicated stages, the embryos were divided into groups, each consisting of 15-20 embryos. Each group was then incubated in a plastic petri dish containing 10 ml of 30% MMR, with or without ethanol for 12 hours. Embryos were allowed to develop for approximately 48 hours at room temperature (22° C.), until the control group reached tadpole stage (stage 45). The conditions used for producing eye defects (Peng et al, 2004), delayed gut development (Peng), microencephaly (Peng et al), and growth retardation (Peng) are shown in detail in the attached references.

Morphological measurements of microcephaly and growth retardation. The tadpoles were then fixed in 2% paraformaldehyde in 30% MMR, and their morphologies examined by light microscopy and photographs were taken at a fixed magnification for body length and head width measurements. The following criteria were recorded: (1) the number of normal/deformed/dead embryos; (2) length of the body measured between the tip of the jaw and the tip of the tail, and (3) width between the eyes measured at the widest level of that of the tadpoles.

Vitamin C treatment. Embryos at stage 13 (initial neural plate stage) were collected and divided into groups, each containing 15-20 embryos and incubated in a plastic petri dish containing MMR medium as stated above. Vitamin C, having a concentration of 100 μM, was added either concomitantly, 2 hours before, or 2 hours after the initiation of a 12-hour alcohol exposure. The tadpoles were fixed and measurements were made as stated above.

Histological analysis of brain injury. Embryos were fixed in 2% paraformaldehyde, dehydrated, embedded in paraffin. The embryos were sectioned coronally through the head and stained with hematoxylin and eosin.

ROS measurement. Embryos were treated with vitamin C 2 hours prior to alcohol exposure, and then transferred to medium in the presence or absence of 0.6% alcohol for 12 hours (from stage 13 until stage 22). At the end of the experiment, embryos were harvested and total ROS content was determined by the calorimetric quantitation of hydrogen peroxide method (total hydroperoxides), following protocols provided by the manufacturer (BIOXYTECH H202-560 kit, OXIS International, Inc. USA).

NF-κB reporter assay. NF-κB luciferase reporter plasmid (pLuc-NF-κB) containing IL-6 promoter region with four repeats of NF-κB binding sites was used to measure NF-κB-mediated transcriptional activity (Li, 1997). Embryos at the two-cell stage were injected with pLuc-NF-κB, treated as indicated in the text, harvested, and lysed at stage 22. Protein concentration was determined. The luciferase activities were measured and calculated as relative luciferase activity per mg of protein.

RT-PCR Animal caps were dissected from embryos at stage 8.5 and cultured in 67% Leibovitz's L-15 medium, pH 7.5 in the presence or absence of varied concentration of alcohol until stage 22. Total RNA was extracted from cultured animal caps with Trizol reagent (Invitrogen). Primer sets and PCR conditions for NCAM and Pax6 have been described in our previous reports (Peng, Neurobiol. Dis., 2004; Peng, J. Mol. Biol., 2004). PCR products were subject to electrophoresis on TBE gel and visualized by ethidium bromide staining. Although data from individual experiments are shown, the results were confirmed in multiple experiments in all cases.

Plasmid construction. Expression plasmid for human catalase (pCMS-CAT) was prepared by inserting the full-length human catalase cDNA into the MCS of the mammalian expression vector pCMS-EGFP (Clontech, Palo Alto, Calif.), which is under the control of the CMV promoter. The expression of EGFP is controlled by a separate SV40-promoter. The expression vector was provided by W. A. Harris and M. E. Zuber at UCSD, USA.

Catalase and Pax 6 Assay: Xenopus laevis embryos were obtained by in vitro fertilization as previously described (Peng et al., 2000). Developmental stages of the embryos were determined using criteria of Nieuwkoop and Faber (Nieuwkoop and Faber, 1994). In each experiment, embryos at 2-cell stage were collected and microinjected with the expression plasmid for either catalase (pCMS-CAT), xPax6 (pCS2+xPax6) or xSox2 (pCS2+xSox2) into each of the two blastomeres. After reaching stage 13, the embryos were divided into groups, each consisting of 20 embryos, which were then incubated in 10 ml of 30% Modified Marc's Ringer (MMR) for 12 hours until stage 22 in the presence or absence of varied concentrations of alcohol as indicated. As alcohol is volatile, the experimental media were replaced with fresh media every 4 hours during the course of alcohol treatment to minimize fluctuations in the alcohol concentration. At stage 22, embryos were transferred to 10 ml of 30% MMR without alcohol and allowed to grow until the control group reached the tadpole stage (stage 40 or 45). The tadpoles were then fixed and examined. Each experiment was repeated at least once with reproducible results.

Animal cap explant assay. Animal caps were dissected from embryos at stage 8.5 and cultured in 67% Leibovitz's L-15 medium, pH 7.5 in the presence or absence of varied concentrations of alcohol until stage 22 (approximately 20 h). Total RNA was extracted from animal caps or whole embryos using TRIZOL reagent (Invitrogen). The total RNA was reverse transcribed using the Superscript system (Invitrogen), and the first strand cDNA of the target gene amplified by PCR. Primers used for PCR are: xOtx-2 (SEQ ID NO:1, 5′-GGATGGATTTGTTGCACCAGTC-3′; and SEQ ID NO:2, 5′-CACTCTCCGAGCTCACTTCTC-3′); xSox2 (SEQ ID NO:3, 5′-GAGGATGGACACTT-ATGCCCAC-3′; and SEQ ID NO:4, 5′-GGACATGCTGTAGG-TAGGCGA-3′); xSox3 (SEQ ID NO:5, 5′-ACCAACACTACCAGAGTGCC-3′ and SEQ ID NO:6, 5′-CCCAACACATCATCCCTACC-3′); xPax6 (SEQ ID NO:7, 5′-CAGAACATCTTTTACCCAGGA-3′ and SEQ ID NO:8, 5′-ACTACTAGGCGA-3′); xNCAM (SEQ ID NO:9, 5′-CACAGTTCCACCAAATGC-3′ and SEQ ID NO:10, 5′-GGAATCAAGCGGTACAGA-3′); xVent-1 (SEQ ID NO:11, 5′-TTCCCTTCAGCATGGTTCAAC-3′ and SEQ ID NO:12, 5′-GCATCTCCTTGGCATATTTGG-3′); xSox17α (SEQ ID NO:13, 5′-GATGGTGGTTACGCCAGCGA-3′ and SEQ ID NO:14, 5′-TGCGGGGTCTGTACTTGTAG-3′); xVegT (SEQ ID NO:15, 5′-CAAGTAAATGTGAGAAACCG-3′ and SEQ ID NO:16, 5′-CAAATACACACACATTTCCC-3′); EF-1α (SEQ ID NO:17, 5′-CAGATTGGTGCTGGATATGC-3′ and SEQ ID NO:18, 5′-ACTGCCTTGATGACTCCTAG-3′).

Western immunoblotting. Stage 25 embryos were lysed with cold PBS with 2 mM phenylmethylsulfonyl fluoride by pipetting. Lysates were resolved on 12.5% SDS-PAGE gels and transferred to Immobilon polyvinylidene difluoride membranes (Millipore). Membranes were probed with a polyclonal antibody against human catalase (Calbiochem, La Jolla, Calif.) and visualized by ECL+Plus reagent (Amersham).

Statistical analysis. Significant differences were determined by using an unpaired Student's t test. For multiple comparisons, the one-way ANOVA with multiple comparisons was used. A p value of less than 0.05 was considered statistically significant.

### Results

The dosage, commencement time, and duration of alcohol treatment required to produce Xenopus embryos with the phenotype of FAS, including significantly smaller body and head size was established. Alcohol exposure conditions in the Xenopus model of the present embodiment produce easily detectable effects on head size and body length and allow efficacy testing of potential protective agents.

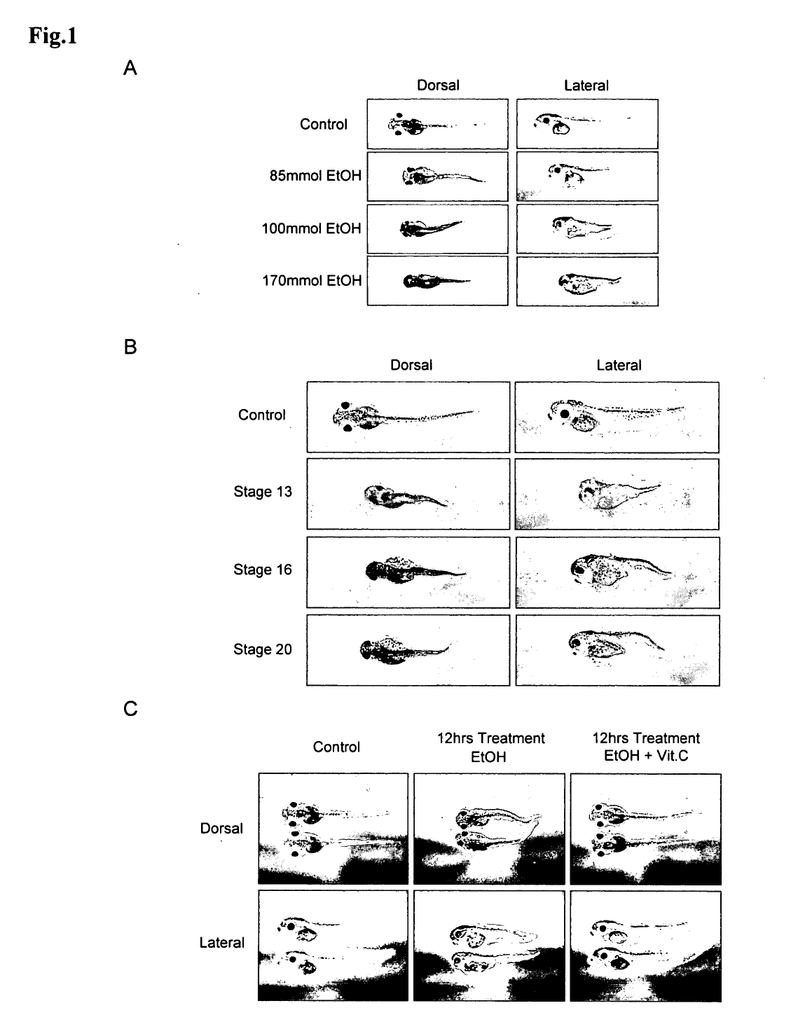

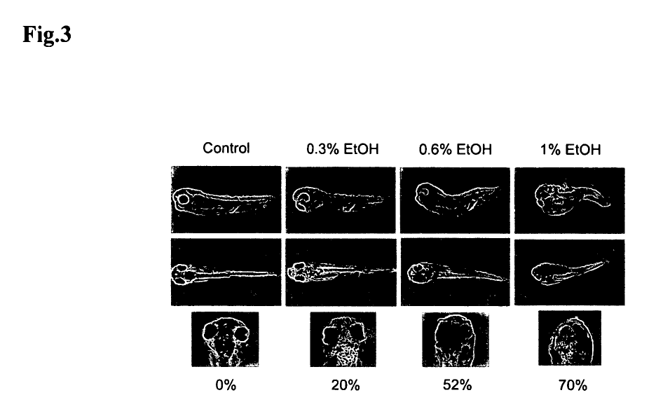

The dosage of ethanol treatment that is required to produce significantly malformed embryos was determined by exposing midgastrulation stage (stage 11) embryos to 30% MMR with 0.3%, 0.4%, 0.5%, 0.6%, 1%, 1.5% or 2% ethanol, allowing them to grow until the control ‘no ethanol’ treatment group reached tadpole stage (stage 45), and examining their morphology. Ethanol exposure caused a dose-dependent increase in the number of dead and deformed tadpoles, and the deformed tadpoles likewise exhibited significant morphological changes (microencephaly and growth retardation as determined by the width between the eyes and the length of the body, respectively; FIG. 3). While embryos were generally resistant to 0.3% ethanol, exposure of embryos to 0.4%, 0.5%, 0.6%, 1%, 1.5% and 2% of ethanol resulted in tadpoles with significantly reduced sizes of the body and head as compared to the control no ethanol treatment group (Table 1 and FIG. 3). In a specific embodiment, embryos are exposed to at least about 0.4%, 0.5%, 0.6%, 0.7%, 0.8%, 0.9% or 1% alcohol. Notably, alcohol treated Xenopus embryos exhibited a variety of growth defects including edema, bended body axis (not shown). However, the neural structures are consistently underdeveloped (FIG. 1A).

TABLE 1
Effects of ethanol dose on the viability and morphology of the
tadpoles^{a}

| | | % body length of | % width between |
|---|---|---|---|
| Ethanol conc | N/def/dead^{b} | control^{c} | eyes of control^{c} |
| 0% | 14/0/1 | 100 ± 19 | 100 ± 12 |
| 0.3% | 11/3/2 | 95 ± 54 | 84 ± 15 |
| 0.4% | 10/4/1 | 90 ± 41 | 81 ± 11* |
| 0.5% | 7/6/2 | 85 ± 57* | 75 ± 09** |
| 0.6% | 5/9/1 | 74 ± 34** | 70 ± 18** |
| 1.0% | 2/10/3 | 70 ± 39** | 69 ± 21** |
| 1.5% | 1/10/4 | 67 ± 51** | 67 ± 17** |
| 2.0% | 0/9/6 | 65 ± 58** | 67 ± 23** |

^{a}Data from representative experiments with 15 embryos per group. Mean and SD are shown as percentage of the average value of the control group.
^{b}N: normal; def: deformed.
^{c}Body length: length of the body of the live tadpoles measured between the tip of the jaw and the tip of the tail; Width/eyes: width between the eyes measured at the widest level.
*P < 0.05 significantly different from the control group which were exposed to no alcohol.
**P < 0.001 very significantly different from the control group which were exposed to no alcohol.

One of the key principles of classical teratology is that the severity of effect on affected organ imposed by an teratogen depends very much on whether or not the organ was exposed to the teratogen during a susceptible stage of development (Hannigan et al., 1999). For the case of alcohol-induced brain injury, it is believed that exposure to ethanol during the brain growth spurt is responsible. In Xenopus, initial neural development is at stage 11, and continues until stage 22.

The window of time during which Xenopus embryos are susceptible to ethanol treatment was determined. Embryos were exposed to 0.6% ethanol at stage 9, 11, 13, 16, 18, 20, 23, 25 and 40 and were grown until the control “no ethanol” group reached tadpole stage. The morphology of the tadpoles was then examined (Table 2). Exposure at initial neural plate stage (stage 13) appears to produce the most severe defects and earlier (from gastrulation, stage 11) or later exposures (later neurula stage, stage 20) both resulted in lesser degree of damage. Embryos before stage 9 or after stage 25 were resistant to the teratogenic effect of alcohol; the tadpoles were not significantly different from that of the control (not shown). When stage 40 tadpoles were exposed to 0.6% alcohol, a high mortality rate (80%) was observed, however, the surviving tadpoles appeared normal. Photos of tadpoles exposed to alcohol at stages 13, 16, and 20 are shown in FIG. 1B. A high percentage of embryos exhibited a bent axis, which is also prominent when embryos were exposed to alcohol at stage 13.

TABLE 2
The severity of damage is related to the developmental stage of initial
ethanol exposure.

| Developmental stage | Normal/deformed/dead |
|---|---|
| No treatment | 20/0/0 |
| Stage 11 | 12/5/3 |
| Stage 13 | 6/12/2 |
| Stage 16 | 8/11/1 |
| Stage 18 | 9/10/1 |
| Stage 20 | 9/9/2 |
| Stage 23 | 15/4/1 |

To determine the minimum duration required to produce fetal deformations, embryos at stage 13 were exposed to 0.6% of alcohol for 1, 2, 4, 6, 12, 24, 36 or 48 hours. The embryos were then transferred to fresh medium and allowed to grow until the control group reached tadpole stage. It appears that exposure of less than 6 hours produced no significant effect in either the viability or the morphology of the tadpoles. A 12-hour exposure (from initial neurula to late neurula, i.e., stage 13-22, consistently showed an increased mortality and marked reduction in head and body sizes, as compared to the control “no ethanol” group (FIG. 1C). Exposure time longer than 24 hours (36 or 48 hours) did not significantly increase the severity of injury (not shown). Thus, in one embodiment, embryos are exposed to alcohol (e.g., 0.6%) for at least about 6, 7, 8, 9. 10, 11 or 12 hours.

Ascorbic acid (Vitamin C) significantly prevented alcohol induced growth retardation and brain injury. Oxidative damage has been suggested to be one of the major mechanisms by which alcohol alters the developing brain (Hannigan, 2000 and Ozer, 2000). The effects of an abundant and safe antioxidant agent, for example, ascorbic acid (Vitamin C), on ethanol-induced defects was tested using the Xenopus model of the present invention. Xenopus embryos were incubated in 100 μM ascorbic acid 2 hours before or after, or concurrently with the commencement of a 12-hour alcohol treatment. It was found that pretreating the embryos with vitamin C significantly reduced the severity of alcohol-induced microencephaly and growth retardation, since the head size and body length of those embryos were restored to near-control values (Table 3, row 5; FIG. 1C).

TABLE 3
The protective effect of vitamin C on ethanol-induced injury

| | | | | Eye | Body |
|---|---|---|---|---|---|
| | No of | | | distance | length |
| Treatment | embryos | Alive | Dead | (mm) | (mm) |
| Control | 15 | 14 | 1 | 2.21 ± 002^{a} | 12.21 ± 013^{a} |
| 0.6% EtOH | 15 | 13 | 2 | 1.63 ± 005^{b} | 9.25 ± 010^{b} |
| VitC alone | 15 | 14 | 1 | 2.19 ± 004^{a} | 12.25 ± 019^{a} |
| VitC + 0.6% | 15 | 14 | 1 | 1.88 ± 003^{a,b} | 10.19 ± 014^{a} |
| EtOH | | | | | |
| Pre-VitC + EtOH | 15 | 14 | 1 | 2.13 ± 002^{a} | 11.88 ± 006^{a} |
| Post-VitC + | 15 | 14 | 1 | 1.82 ± 004^{b} | 9.60 ± 013^{b} |
| EtOH | | | | | |

Values represent mean ± SD. Significant difference was analyzed using one-way ANOVA with multiple comparisons.
^{a}P < 0.001 significantly different from the 0.6% EtOH-treated group.
^{b}P < 0.001 significantly different from the control no-ethanol group.

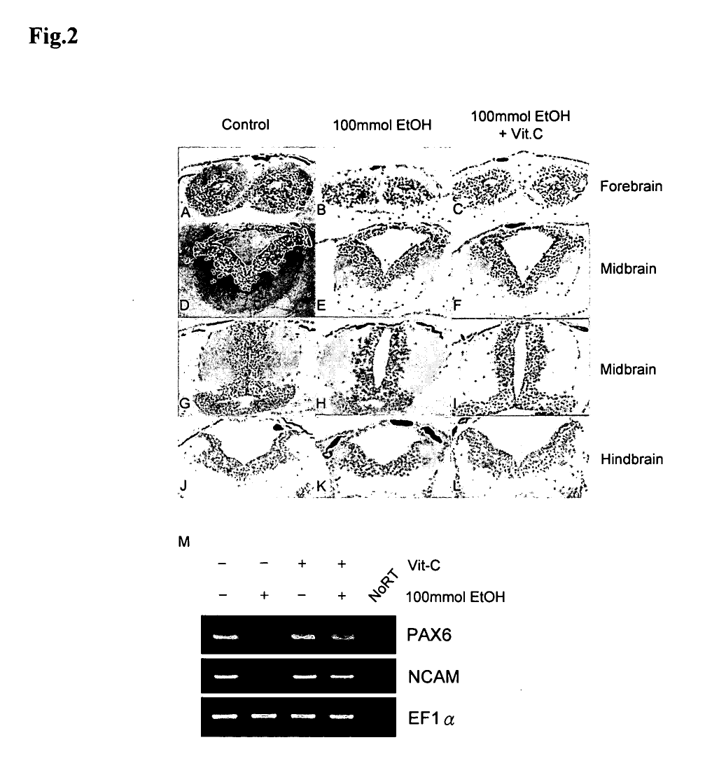

Histological examination of the tadpoles at stage 45 revealed that addition of vitamin C at 2 hours before the addition of alcohol showed a significant restoration of the forebrain areas (FIGS. 2A through 2L). There was a reduction in size of the forebrain area in the tadpoles treated with alcohol when compared to the control (compare FIGS. 2A and 2B). However, addition of vitamin C before or concomitantly with the alcohol could mitigate the injurious effects of the alcohol (FIG. 2C). The sizes of the grey and white matter showed no significant difference from the control. On the other hand, in the diencephalon areas (FIGS. 2D through 2I), the effect of alcohol was not so severe. As shown in FIG. 2 D-F, the areas of grey and white matter and the morphology of the neuronal cells were not changed dramatically. Only a change in the width of the canal was found in alcohol-treated tadpoles as compared to the control (compare FIGS. 2G and 2H). Addition of vitamin C concomitantly could not narrow down the canal (FIG. 2I). In the tectum areas (FIGS. 2J through L), the size of the alcohol-treated tadpoles was not decreased significantly, nor was the size of the grey and white matter. However, the sizes of the neuronal cells appeared to be larger than the control. Addition of vitamin C concomitantly could reduce the effect by alcohol. Consistent with these morphological observations, the expression of a pan-neural marker NCAM and neural and eye marker (Pax6) was also restored significantly by vitamin C pretreatment (FIG. 2M). Vitamin C alone did not have any adverse effect on the brain development. The EF-1α transcript was detected as an internal control for equal RNA load.

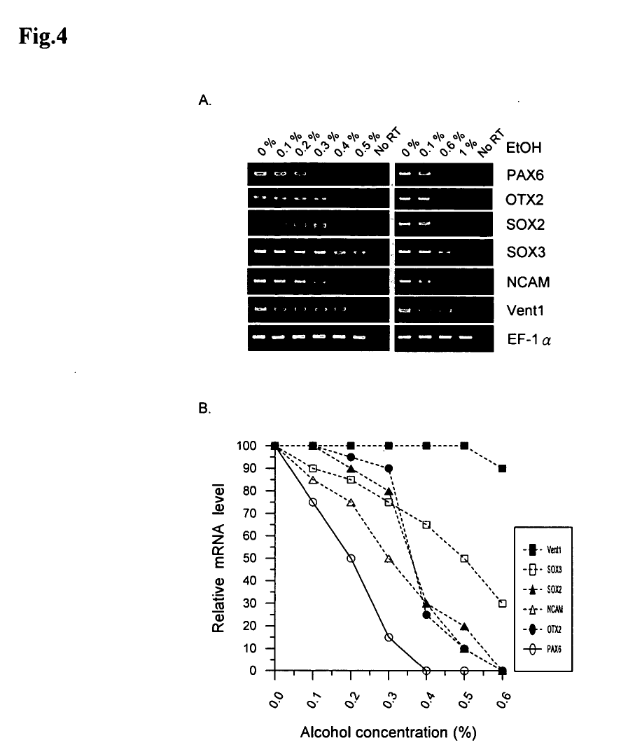

Alcohol exposure reduces the expression of neural markers. In this embodiment, the mechanism by which alcohol induces changes in neural development was elucidated using the model system of the invention and the animal cap assay to further examine the effect of alcohol on the expression of several molecular neural markers. The Xenopus animal cap is composed of pluripotent cells that can be induced to form neural, mesodermal, or epidermal tissue, and is a useful tissue to assess the activity of various factors on neural development. Animal caps were treated with varied concentrations of alcohol as indicated. As shown in FIG. 4, at concentrations ranging from 0.1% to 0.6%, alcohol dose-dependently inhibited the expression of Pax6 (neural and eye marker), NCAM (pan-neural marker), Otx2 (anterior neural region marker), Sox2 (pan-neural marker), and Sox3 (prospective neuroectoderm marker), of which Pax6 was the most vulnerable. The expression of Pax6 was suppressed by 25%, 50%, and 90% at an alcohol concentration as low as 0.1%, 0.2% and 0.3% (65 mM), respectively. NCAM expression was reduced by 15%, 25%, 50%, and 70% at 0.1%, 0.2%, 0.3%, and 0.4% alcohol, respectively. By contrast, Otx2, Sox2, and Sox3 expression was less sensitive to alcohol and the expression of the general ventral marker, Vent1, was not seriously affected.

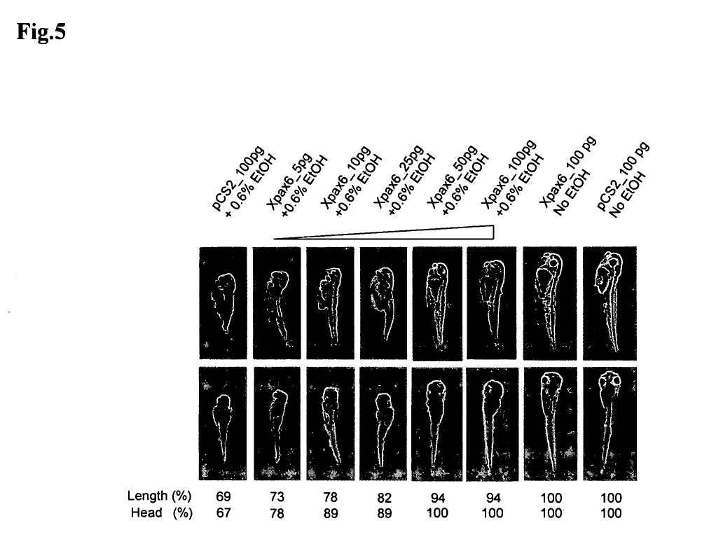

Ectopic expression of xPax6 is sufficient to rescue alcohol-induced microcephaly To test whether alcohol-mediated the suppression of Pax6 is the critical factor for microcephaly, a rescue experiment was conducted. Embryos were injected with the control vector or varied concentrations (0, 5, 10, 25, 50, and 100 pg) of the xPax6 expression plasmid pCS2+xPax6 (Henderson et al., 1999) into two blastomeres at two-cell stage and exposed to either control medium or 0.6% alcohol for 12 hours (from stage 13 to stage 22). The injected embryos were allowed to grow to stage 40, and their head size and body length were then compared to that of the control embryos injected with mock plasmid pCS2+ and without alcohol treatment. Expression of xPax6 or mock plasmid alone had no effect on embryonic development. Furthermore, ectopic expression of xPax6 dose-dependently restored alcohol-induced microcephaly and growth retardation (FIG. 5). Maximal protection was observed in the 50 and 100 pg xPax6 treatment groups where the average head size of the tadpoles was not significantly different from that of the control group. The body length, however, was only partially restored to an average of 94% of the control group (n=50, p<0.05, significantly different from the control group). In a separate experiment, 100 pg pCS2+xSox2 or pCS2+ plasmid was injected into animal pole at two-cell stage embryos (n=50) and treated these embryos in 0.6% alcohol under the same conditions as described for Pax6. Over-expression of Sox2 did not significantly prevent microcephaly or growth retardation (data not shown). The frequency of noted microcephaly and growth retardation for tadpoles injected with pCS2+xSox2 was 48% as compared to 52% in pCS2+ injected group. Furthermore, there is no significant difference in head size and body length in the deformed tadpoles (data not shown) These results suggest that, in contrast to Pax6, ectopic expression of Sox2 could not significantly prevent alcohol-induced microcephaly and growth retardation.

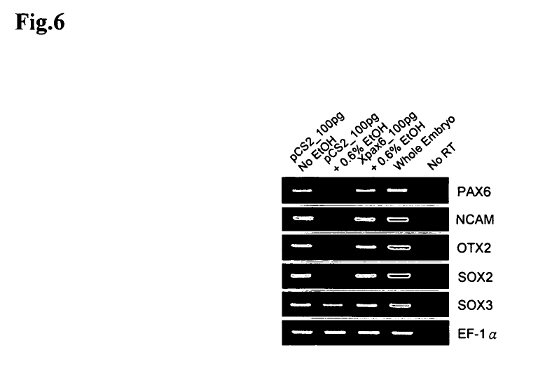

The effect of xPax6 on the expression of other neural markers was then examined using the animal cap assay. Embryos were microinjected with 100 pg of either pCS2+xPax6 or the control plasmid pCS2+. At stage 8.5, animal caps were dissected, and then incubated in either control media or media containing 0.6% alcohol until stage 22. Microinjection of the pCS2+xPax6 plasmid restored the expression of xPax6 gene. Moreover, it also restored the expression of other neural markers Otx2, Sox2, Sox3, and NCAM to nearly normal level (FIG. 6), suggesting that suppression of Pax6 is the upstream event. Together with the morphological measurements, these results indicated that Pax6 is a critical target of alcohol and a key regulator of neural development such that expression of xPax6 is sufficient to prevent alcohol-induced microcephaly and reduce neural gene expression and could be expressed as a treatment. This data also showed that growth retardation was not fully rescued by xPax6, suggesting that additional pathways may be involved in alcohol-induced growth retardation.

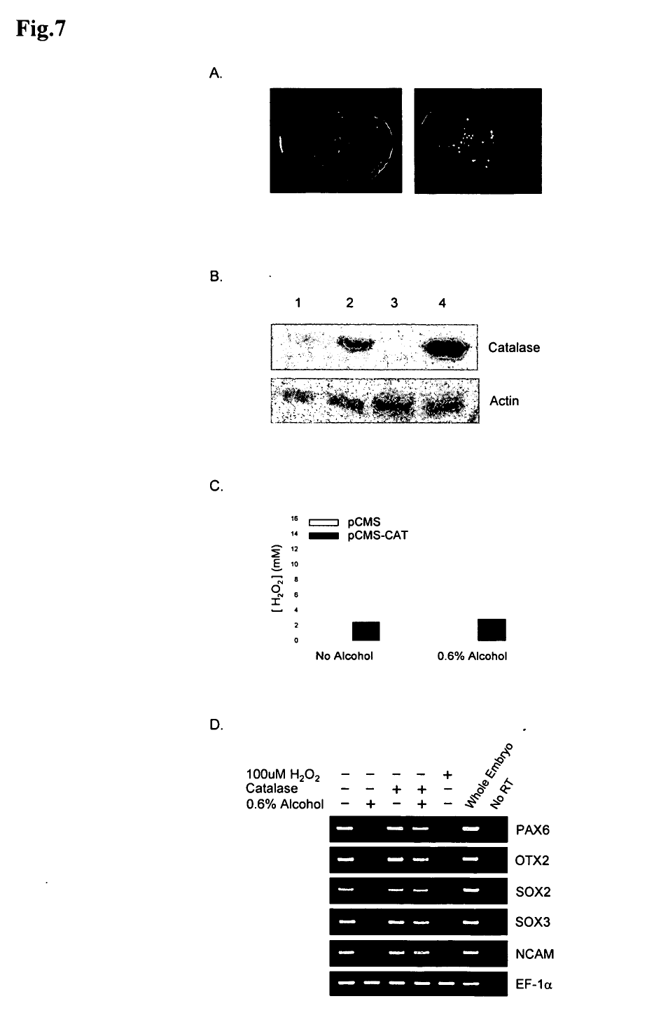

Ectopic overexpression of catalase prevents alcohol-induced ROS production and microcephaly in developing embryos. Oxidative stress caused by reactive oxygen species (ROS) has been proposed to be the causal agent for FAS. As H₂O₂ is the major ROS produced by fetal alcohol exposure (Ozer et al., 2000), whether eliminating alcohol-induced H₂O₂ production can prevent microcephaly was examined by the protective effect of catalase. Human catalase was overexpressed in Xenopus embryos by microinjecting the two-cell stage embryos with 100 pg of catalase expression plasmid pCMS-CAT, and then treating them with or without 0.6% alcohol from stage 13 to 22. The expression of catalase was confirmed by the observation of the EGFP fluorescence at stage 22 (FIG. 7A) and by Western immunoblot analysis of the catalase protein at stage 40 (FIG. 7B). To demonstrate the in vivo production of ROS, embryos were harvested at stage 22 for the analysis of H₂O₂. These results showed that alcohol significantly augmented in vivo H₂O₂ production, which could be effectively abrogated by the expression of catalase (FIG. 7C) and suggest treatment options in accordance with this embodiment.

More importantly, catalase alleviated alcohol-induced microcephaly. Microinjection of 100 pg expression plasmid for catalase restored the head size from an average of 81% to 98% of the control group (not significantly different from that of the control), indicating that the enzyme protects the embryos from alcohol-induced microcephaly. Similar to the effect of xPax6, catalase offered less protective effect to growth retardation because it only restored the body length from an average of 84% to 93% of the control (Table 1).

TABLE 4
Effect of catalase on alcohol-induced injuries.

| | | | Distance |
|---|---|---|---|
| | | Body length | between eyes |
| Treatment | Total/alive | (% of control) | (% of control) |
| pCMS-EGFP | 40/32 | 100.0 ± 5.8^{b} | 100.0 ± 5.1^{b} |
| pCMS-CAT | 40/31 | 102.0 ± 5.2^{b} | 97.8 ± 5.8^{b} |
| pCMS-EGFP + alcohol | 40/32 | 83.7 ± 10.1^{a} | 81.3 ± 14^{a} |
| pCMS-CAT + alcohol | 40/31 | 93.1 ± 7.4^{a,b} | 98.2 ± 2.7^{b} |

Data are combined from two experiments, with 20 embryos per group for each experiment. Mean and SD are shown as percentage of the average of the control group. Body length and eye distance were measured as described in Methods. Values are given as mean ± SD.
^{a}p < 0.05, significantly different from that of the control pCMS-EGFP group.
^{b}p < 0.05, significantly different from that of the pCMS-EGFP + alcohol group.

Catalase restores the expression of Pax6 and other neural genes. The Xenopus animal cap assay was used to determine whether catalase exerts its protective effect by modulating the Pax6 signaling pathway. Microinjection of catalase expression plasmid alone has no effect on the expression of Pax6, Otx2, Sox2, Sox3, and NCAM. On the other hand, catalase restored alcohol-induced suppression of Pax6, Otx2, Sox2, Sox3, and NCAM gene expression to a level comparable to that of the control group without alcohol treatment (FIG. 7D). To test whether the effect of alcohol can be mimicked by ROS delivery, animal caps were also treated with 100 M of H₂O₂ instead of alcohol. We showed that H₂O₂ also reduced the expression of Pax6, Otx2, Sox2, Sox3, and NCAM gene expression (FIG. 7D). Taken together, these results indicate that ROS-mediated suppression of Pax6 gene expression plays a pivotal role in alcohol-induced microcephaly. This is particularly significant, as discussed above, because Pax6 is most vulnerable and one of the most important alcohol targets. The differential protective role of catalase on microcephaly versus growth retardation also suggests that oxidative stress may only be one of the mechanisms that underlie alcohol-induced abnormalities.

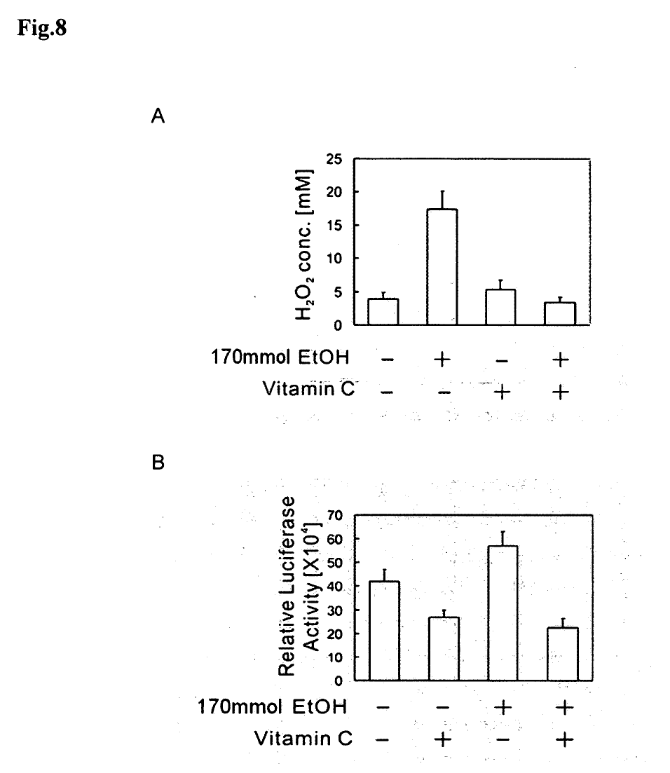

Vitamin C inhibited alcohol-induced ROS as well as NF-κB activation. In another embodiment, the mechanism by which vitamin C protects the developing brain against alcohol induced damage is shown, and the mechanism of reactive oxygen species as mediators of alcohol-induced brain injury was measured by the level of hydrogen peroxide in the embryos. FIG. 8A indicates the hydrogen peroxide level in the embryos significantly increased in the presence of ethanol. This upshot of hydrogen peroxide was suppressed when the embryos were pretreated with vitamin C before ethanol treatment. Taken together with the phenotypic observations, vitamin C appears to prevent the production of ROS induced by ethanol. This downregulation of ROS may be responsible in part for the neural protective activity of vitamin C.

NF-κB is a nuclear transcription factor which normally exists in an inactive form bound by IκB in the cytoplasm. Upon activation, NF-κB dissociates with IκB and translocates into the nucleus where it activates transcription. Ethanol has been associated with NF-κB activation in the naive rat brain (Ward et al, 1996). However it has not been shown before whether NF-κB activation takes any role in alcohol-induced brain injury and growth retardation. In this embodiment, NF-κB activity was measured in the whole embryo by the reporter construct pLuc-NF-κB, which was injected into embryos at 2-cell stage. Injected embryos were treated with 0.6% ethanol as described above, and the activity of luciferase r.

Reporter recorded when the embryos reached stage 22. Upon ethanol insult, NF-κB activity in the embryo increased. This increase in NF-κB activity is suppressed when embryos are pretreated with vitamin C, indicating that alcohol induces NF-κB activation in whole embryos in a reactive oxygen species dependent manner (FIG. 8B).

TABLE 5
Comparison of the gut developmental stage and body length in tadpoles
with and without alcohol exposure.

| | Number of embryos in | | | | | Body length |
|---|---|---|---|---|---|---|
| | gut developmental stages | | | | | (% no |
| Treatments | 42 | 43 | 44 | 45 | 46 | alcohol control) |
| No alcohol | 0 | 0 | 5 | 8 | 7 | 100 ± 2.2 |
| 0.5% alcohol | 3 | 8 | 7 | 2 | 0 | 87 ± 2.7 |
| pCMS | 0 | 0 | 2 | 7 | 11 | 100 ± 2.2 |
| pCMS + 0.5% alcohol | 3 | 7 | 9 | 1 | 0 | 85 ± 3.1 |
| Catalase | 0 | 0 | 5 | 8 | 7 | 99 ± 2.7 |
| Catalase + 0.5% alcohol | 2 | 5 | 10 | 3 | 0 | 88 ± 5.1 |
| cpPRDX5 | 0 | 0 | 4 | 7 | 9 | 99 ± 4.1 |
| cpPRDX5 + 0.5% alcohol | 0 | 5 | 6 | 7 | 2 | 92 ± 2.1 |
| mtPRDX5 | 0 | 0 | 4 | 6 | 10 | 101 ± 3.2 |
| mtPRDX5 + 0.5% alcohol | 0 | 1 | 6 | 5 | 8 | 93 ± 2.7 |

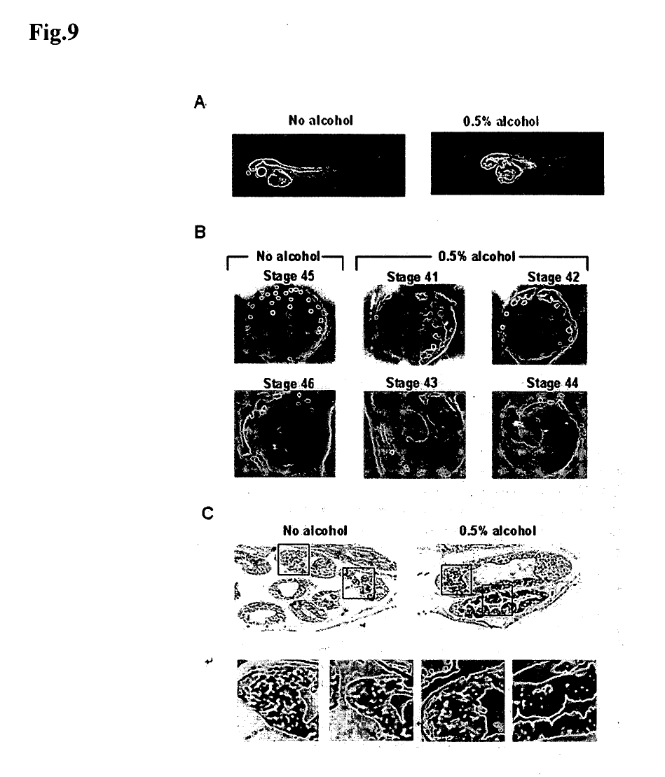

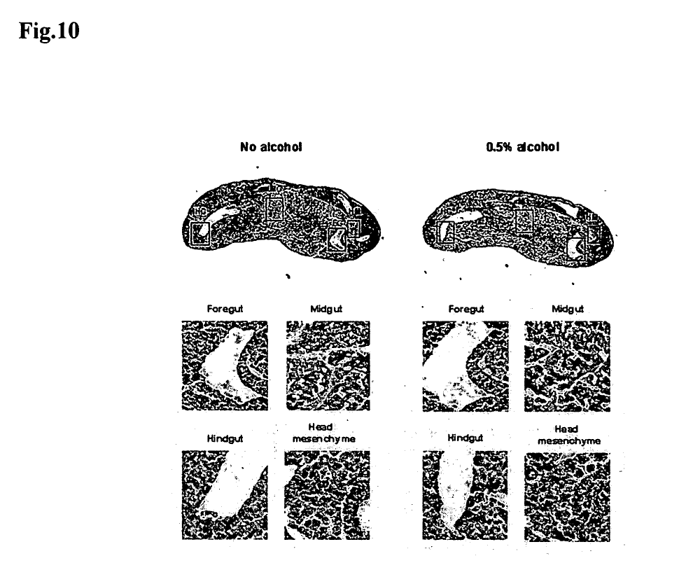

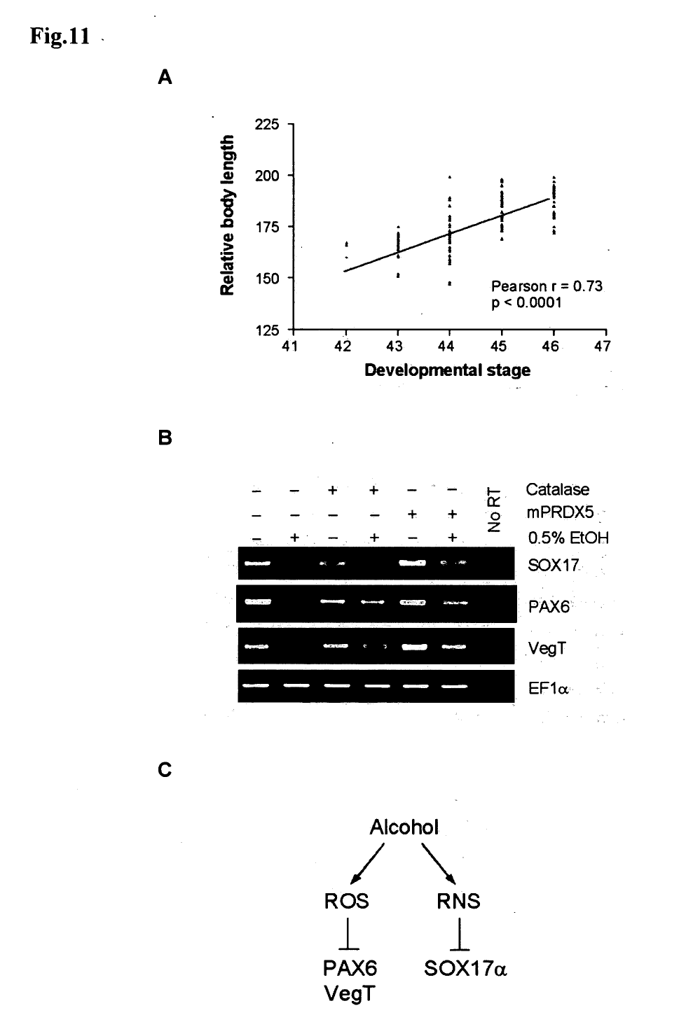

The relevance and applicability of this model to human FAS are suggested by the concentration of alcohol (0.5%) capable of producing the delayed gut maturation and growth retardation. Exposure of embryos to 0.5% alcohol between stage 13 to stage 22 (approximately 12 hours) produced tadpoles with microcephaly¹¹ (smallness of the head), ocular anomalies (incomplete closure of the choroid fissure),¹² growth retardation (Table 5, FIG. 9A), as well as a marked delay in gut development (FIG. 9B). It was well documented by Chalmers and Slack¹³ that a normal Xenopus gut at stage 42 embryos is only a short thick tube. It is then transformed into a long thin tube displaying definite looping pattern at different stages. At stage 46, the gut is composed of distinct stomach, proximal small intestine, external coil of small intestine, internal coil of small intestine, internal coil of large intestine, and distal large intestine. As shown in FIG. 9B and Table 5, transient alcohol exposure between stages 13-22 produced a significant delay in gut maturation which can be determined by examining the looping pattern of the tadpole gut. While the gut of all of the control tadpoles reached developmental stages 45-46, the gut of the 0.5% alcohol-treated tadpoles was still at developmental stages 42-44 (Table 5, FIG. 9B). Morphologically, the alcohol-treated group showed a clear reduction in the number of loops formed in the intestine (FIG. 9B). In order to test the morphological effect of alcohol in different parts of the gut, we have performed H&E staining on sagittal sections of the control and alcohol-treated embryos at both the tadpole stage (developmental stages 42-46, FIG. 9C) and the tailbud stage (stage 23, FIG. 10). At the tadpole stage, the alcohol-treated group had a small number of intestinal loops and an abnormally large gut lumen when compared with the control. However, we did not observe any morphological effect of alcohol in specific regions of the gut (foregut, midgut or hindgut) or in the head mesenchyme of stage 23 tailbuds.

The amphibian intestine goes through extensive remodeling during metamorphosis. The morphology of the gut during Xenopus metamorphosis has been described in details by Sundqvist and Holmgren.¹⁴ Briefly, at prometamorphosis (NF stages 56-59), the approximate length of the tadpole gut is 150 mm. At NF stages 61-62, the tadpole is approximately ⅔ shorter, at NF stages 65-66, the gut is 15-20 mm. These changes can be easily distinguished by observing the gut morphology. For this reason, we followed the gut morphology of the alcohol-treated and control tadpoles from stage 42 up to stage 65. We observed significant differences in the looping pattern at the time when normal tadpoles were at stages 45-46 (FIG. 9B and Table 5). Afterwards, the differences became less prominent. When the gut was examined at stage 60, no difference can be found between alcohol-treated and control groups. These results suggest that transient alcohol exposure caused a delay in gut development during critical development stages, and that this delay is reversible upon alcohol withdrawal. In contrast, a sustained alcohol exposure of alcohol from stage 13 caused the death of tadpoles at around stages 45-50.

To delineate the causal roles of ROS and RNS in delayed gut development and growth retardation, we determined the protective effects of catalase and both mtPRDX5 and cpPRDX5 in vivo.^{20,21} Control vector or expression plasmids for mtPRDX5, cpPRDX5, and catalase were injected into the two blastomeres of 2-cell stage embryos, and the embryos were treated with media containing either 0% or 0.5% alcohol between stages 13 and 22. Over-expression of all three enzymes provided a varying degree of protection against alcohol-induced delayed gut development and growth retardation in which mtPRDX5 offered most protection (Table 5). We also examined the correlation between the stage of gut development and body length in each individual tadpole. A significant positive correlation was found (r=0.73), suggesting a cause-and-effect relationship between delayed gut development and growth retardation (FIG. 10A).

Besides performing these morphological studies, we also examined the effect of catalase and mtPRDX5 on the expression of key gut developmental genes by animal cap explant assay. As shown in FIG. 10B, alcohol suppressed the expression of Sox17, Pax6 and VegT. Microinjection of catalase alone did not have any effect. However, it rescued alcohol-mediated suppression of inhibition of Pax6 and VegT, but not Sox17 gene expression. By contrast, mtPRDX5 not only restored the expression of Pax6, and VegT, but also the Sox17 gene. These results suggest that ROS and RNS represent two separate mechanisms for alcohol-induced delayed gut development, which inhibit either Pax6/VegT or Sox17 respectively (FIG. 10C).

Based on the observations described above, the present invention also provides a method for screening an agent which is protective or therapeutically useful, i.e., for preventing or treating FAS, using the Xenopus laevis embryo model of the present invention. The reduction of birth defects by such an agent may be detected by a variety of methods, including, but not limited to, observing morphological changes, and measuring the levels of neural marker expressions in the animal cap of the embryo, e.g., expression of Pax6, Sox2, Sox3, and/or NCAM.

Furthermore, the present invention provides a method for treating FAS by administering an agent which causes or enhances an expression of Pax6. Such an agent includes, but not by way of limitation, vitamin C, catalase, or other agents which can be selected by using the screening methods described above. In another embodiment, the invention provides a method for treating FAS by administering an agent which causes suppression of NF-κB activation. Such an agent includes vitamin C and/or various agents selected by the screening methods of the present invention.

### Discussion

Xenopus as an animal model of fetal alcohol syndrome: In this study, it was demonstrated that exposure of Xenopus embryos to ethanol between initial neural plate stage to late neural stage is required and sufficient to produce the small head/body phenotypes similar to those observed in human and mouse FAS. This result is consistent with a previous report, which shows that exposure of Xenopus embryos to ethanol from early gastrulation stage to the late neurula stage produced tadpoles with significantly smaller head (Nakatsuji, 1983). Using the Xenopus model of the present invention, the window of alcohol exposure required was narrowed. The observations in the Xenopus model are also consistent with the published data in mouse (Olney, et al., 2002) and chicken (Natsuki, 1990) models of FAS.

The ethanol effect is readily reversible and does not produce significant effects on tadpoles when ethanol is either removed prior to the mid-neurula stage or administered after late-neurula stage. These results suggest that the changes acquired during the initial to late neurula stage are critical, and are probably the proximal causes of FAS, and that there is a window of opportunity for intervention.

Antioxidants can protect against alcohol-induced brain injury. According to the present invention, the antioxidant vitamin C can reduce the severity of alcohol-induced brain injury and growth retardation. It is very likely that vitamin C exerts its neuroprotective effect by reducing the ROS level in alcohol treated embryos. In a rat model, the amount of ethanol-mediated oxidative stress was mitigated when pregnant rats were treated with folic acid concomitantly to ethanol administration (Cano, et al., 2001). The antioxidant capacity of folic acid seems to be involved in its protective effect. Vitamin E or β-carotene protected embryonic hippocampal cultures against 400-2400 mg/dl of ethanol treatment (Mitchell, et al., 1999). Taken with our study, it appears that there is a possibility for nutritional therapies incorporating antioxidants to help protect against deleterious fetal effects from maternal alcohol abuse.

Alcohol and NF-κB. Ethanol was shown to be able to modulate NF-κB activity in different biological settings, through pathways involving reactive oxygen species. Hence, addition of antioxidant agents can abrogate the effect of ethanol on cellular NF-κB activity (Altura, 2002 and Toldano, 1991). Further evidence on this was obtained using the Xenopus model of the present invention, since pretreating Xenopus embryos with the antioxidant vitamin C significantly reduced hydrogen peroxide production, as well as NF-κB activation, induced by ethanol.

Modulation of NF-κB activity by ethanol is tissue specific. Generally, ethanol down-regulates or does not exert direct effect on NF-κB in the immune system, but does activate NF-κB in other tissues. For instance, ethanol on its own cannot activate NF-κB in T-lymphocytes but can potentiate NF-κB activation by TNF-alpha, in a ROS-dependent manner (Dong et al., 2000). In monocytes ethanol interferes with inflammatory cytokine production by down-regulating NF-κB, resulting in impaired immunity (Mandrekar, 1997 and Mandrekar, 1999). In contrast, ethanol is a potent inducer of lipid peroxidation and activation of NF-κB in cerebral vascular muscle cells (Altura, 2002). Ethanol may also contribute to bone loss through activation of NF-κB signal transduction that results in production of an osteroclastogenic cytokine in osteoblasts (Yao et al., 2001). In addition, NF-κB induction could be detected in the brains of naive rats upon acute injection of ethanol (Ward et al., 1996).

For the first time, the possible involvement of NF-κB activation in alcohol-induced brain development injury was demonstrated using the Xenopus model according to the present invention. NF-κB activation induced by alcohol may be mediated by the ROS generated upon treatment.

Use of the Xenopus model of the present invention may provide insight into the identification of the important genes controlling neurogenesis and growth. Pax6 is identified as a crucial target for the detrimental effects of alcohol on fetal development, as overexpression of Pax6 effectively rescues alcohol-induced microcephaly. Furthermore, the facility of conducting experiments using the Xenopus embryo makes it an excellent model system to investigate and elucidate the molecular basis of alcohol-induced disorders, and to develop an effective neuroprotective therapy.

Oxidative stress-mediated suppression of Pax6 is a critical target for alcohol-induced microcephaly and is partly responsible for alcohol-induced growth retardation. Reactive oxygen species (ROS)-induced oxidative stress has long been linked to FAS (Guerri et al., 1994; West, 1994; Henderson et al., 1995; Henderson et al., 1999). Studies conducted in vitro in brain-derived cells showed that alcohol stimulates the production of ROS, which includes oxygen radicals such as superoxide (O2-) and hydroxyl radicals (.OH), and non-radicals like H₂O₂. These highly reactive and unstable species are thought to exert their deleterious effects primarily by damaging virtually all classes of biomolecules including DNA, protein, lipid, sugars, and polysaccharides (Henderson et al., 1999), leading to impaired signaling and eventually cell death. Results from this study and according to the various embodiments of the invention, show the possibility that ROS can cause brain damage through modulating the expression of key genes for neural development. ROS can either perturb the transcription of these genes or inhibit the signaling pathways for neurogenesis.

The brain is exceptionally vulnerable to oxidative damage because the brain has a high level of oxygen consumption, and yet its antioxidant defense mechanisms are not particularly well-developed. For example, the catalase activity in the brain is only about 10% of that in the liver (Marklund et al., 1982). Moreover, the brain is enriched in polyunsaturated fatty acids, which are good substrates for lipid peroxidation (Floyd and Carney, 1992). Therefore, it is conceivable that alcohol may produce microcephaly in Xenopus embryos by increasing the concentration of ROS within the brain, which in turn suppresses the expressions of key regulatory genes and thus affects fetal development.

Catalase catalyzes the breakdown of H₂O₂ to O₂ and H₂O and offers protection against the toxic effects of oxygen radicals. This enzyme is ubiquitously expressed in the adult tissues. As a natural protective mechanism, its expression is induced by alcohol and hyperoxia condition in the rat embryos (Aspberg and Tottmar, 1994; Mover and Ar, 1997). Catalase was previously shown to be able to support the survival of embryonic CNS neurons in vitro by blockading free radical formation (Walicke et al., 1986; Martin et al., 1987; Chau et al., 1988). Similarly to Pax6, overexpression of catalase reduces the frequency of alcohol-treated embryos with microcephaly, but it cannot fully prevent growth retardation. The reason for this is at present unknown. Nevertheless, according to the invention, catalase may reverse the teratogenic effects of alcohol by reducing the levels of H₂O₂ in vivo and by normalizing Pax6 expression and could be used as a treatment.

This report is the first to directly delineate the cause-and-effect relationship of ROS and alcohol-induced injuries. We demonstrated the protective effect of catalase against alcohol-induced H₂O₂ production as well as alcohol-induced microcephaly and growth retardation. More importantly, the linkage between H₂O₂ and Pax6 signaling, was shown by the animal cap assay in which 100M of H₂O₂ mimics the effect of alcohol by suppressing the expression of Pax6 and other neural genes.

Neural development consists of several major processes including neural initiation, maintenance, differentiation, etc. Neural initiation occurs as early as at stage 10, after that other processes gradually join the mission and co-exist until the whole nervous system is built up. Pax6 is a homeodomain-containing transcription factor which is important for the development of the brain, spinal cord, eye, nose, pituitary, and pancreas (Callaerts et al., 1997). In the CNS, it has been implicated in brain patterning, neuronal specification, neuronal migration, and axonal extension (Osumi, 2001). It also acts as a control switch for glial-neuronal trans-differentiation (Heins et al., 2002). The crucial roles of Pax6 in brain development are substantiated by homozygous Pax6 mutant mice which display severe abnormalities of the CNS, including forebrain patterning defects (Stoykova et al., 1996) and deformed cerebral cortex (Gotz et al., 1998; Pratt et al., 2000). In accordance with embodiments of the invention, alcohol was shown to downregulate Pax6 expression and induce microcephaly in Xenopus embryos, which is consistent with the pivotal functions of Pax6 in brain development.

Sox2, a Sry-related HMG factor, is insufficient to cause neural differentiation, but works as a cofactor to help the ectodermal cells strengthen their neural fate and further respond to extracellular signals (Peng et al., 2002). Consistently, these results showed that ectopic expression of Sox2 is not sufficient to prevent alcohol-induced microcephaly and growth retardation, but ectopic expression of same dose xPax6 alone restored alcohol-induced microcephaly and rescured partly growth retardation (FIG. 5). Like Sox2, the other neural marker Sox3, Otx2 and NCAM expression was less sensitive than Pax6 to alcohol. Based on these results, Pax6 is one of the most important alcohol targets, although other genes might also be involved in the microcephaly phenotype.

Sox17, a Sry-related HMG box transcription factor, functions as an endoderm determinant or an obligate mediator of vertebrate endoderm development.²³ Pax6 has a diverse role in the development of the neural tissues and the eyes, and also plays a critical role in the development of duodenal GIP cells and the gastrin and somatostatin cells of the distal stomach.²⁴ VegT is a maternal T-box transcription factor required to initiate endoderm formation. It functions near the top of an endoderm-specifying transcriptional hierarchy that activates and reinforces Nodal-related TGFbeta signaling. It also induces the expression of essential downstream transcriptional regulators of the endodermal genes Bix1, Bix3, Bix4, Milk, Mix.1, Mix.2, Mixer, Xsox17 alpha, Gata4, Gata5, Gata6 and endodermin, the anterior endodermal genes Xhex and Cerberus, and the organizer specific gene Xlim1.²⁵ Based on the critical roles of Sox17, Pax6, and VegT in embryonic development, we speculate that the downregulation of these genes is largely responsible for the alcohol-induced delayed gut development and growth retardation observed in our Xenopus model.

It has been suggested that alcohol promotes oxidative and nitrosative stresses on the tissues by increasing the production of ROS and RNS.^{17, 19, 26} ROS encompasses oxygen radicals such as superoxide (.O₂—) and hydroxyl (.OH), and non-radicals like hydrogen peroxide (H₂O₂) and ozone (O₃), whereas RNS includes a number of highly reactive nitrogen-containing radicals and non-radicals such as nitric oxide (.NO), nitrogen dioxide (.NO₂), nitrous acid (HNO₂), and peroxynitrite (ONOO⁻). Both ROS and RNS are powerful oxidizing agents. When present in excess, these reactive species can overwhelm the cellular antioxidant defense mechanisms, leading to cell apoptosis, chromosome aberration, lipid peroxidation, and protein damage. The results of this study suggest that ROS and RNS may also affect gene expression. High levels of ROS and RNS during gestation may disturb the general intrinsic mechanisms of gene expression and compromise the dynamics of enteric tissue development. We show that alcohol specifically suppresses the expression of a VegT, Pax6, and Sox17, but has a much less significant effect on other gut developmental genes. This raises the possibility that alcohol induces the downregulation of a special subset of gut developmental genes. Furthermore, while ectopic expression of catalase only restores the expression of VegT/Pax6, PRDX5 can restore the expression of VegT/Pax6 as well as Sox17. This suggests that oxidative stress and nitrosative stress may target different genes.

The Xenopus model, with its well-defined developmental stages, in vitro culture, the rapid rate of development and ease of manipulations, appears to be an excellent model to study the molecular basis for FAS, with the limitation of cortical migration anomalies of the mammalian cortex that Xenopus laevis is unlikely to show since the Xenopus “cortex” is much more simply organized than the mammalian cortex.

The processes and devices described above illustrate preferred methods and typical devices of many that could be used and produced. The above description and drawings illustrate embodiments, which achieve the objects, features, and advantages of the present invention. However, it is not intended that the present invention be strictly limited to the above-described and illustrated embodiments. Any modifications, though presently unforeseeable, of the present invention that come within the spirit and scope of the following claims should be considered part of the present invention.

### REFERENCES

The following citations are hereby incorporated by reference in their entireties for details supplementing this application:
[1] Y. Peng, K. H. Kwok, P. H. Yang, S. S. Ng, J. Liu, O. G. Wong, M. L. He, H. F. Kung, M. C. Lin, Ascorbic acid inhibits ROS production, NF-kappa B activation and prevents ethanol-induced growth retardation and microencephaly. Neuropharmacology. 2005, 48(3):426-34.
[2] Y. Peng, P. H. Yang, S. S. Ng, O. G. Wong, J. Liu, M. L. He, H. F. Kung, M. C. Lin., A critical role of Pax6 in alcohol-induced fetal microcephaly, Neurobiol Dis., 2004, 16(2):370-6.

[3] Y. Peng, P. H. Yang, Y. Guo, S. S. Ng, J. Liu, P. C. Fung, D. Tay, J. Ge, M. L. He, H. F. Kung, M. C. Lin. Catalase and peroxiredoxin 5 protect Xenopus embryos against alcohol-induced ocular anomalies. Invest Ophthalmol Vis Sci. 2004; 45(1):23-9.
[4] Y. Peng, P. H. Yang, S. S. Ng, C. T. Lum, H. F. Kung, M. C. Lin., Protection of Xenopus laevis embryos against alcohol-induced delayed gut maturation and growth retardation by peroxiredoxin 5 and catalase, J. Mol. Biol., 2004, 340(4):819-27.
[5] Y. Peng, K. H. Kok, R. H. Xu, K. H. Kwok, D. Tay, P. C. Fung, H. F. Kung, M. C. Lin, Maternal cold inducible RNA binding protein is required for embryonic kidney formation in Xenopus laevis, FEBS Lett., 2000, 482:37-43.
[6] P. D. Nieuwkoop and J. Faber, Normal table of Xenopus laevis (Daudin): a systematical and chronological survey of the development from the fertilized egg till the end of metamorphosis, Garland Pub, New York, 1994.
[7] J. J. Li, C. Westergaard, P. Ghosh, N. H. Colburn, Inhibitors of both nuclear factor-kappaB and activator protein-1 activation block the neoplastic transformation response, Cancer Res., 1997, 57:3569-76.
[8] J. H. Hannigan, L. P. Spear, N. E. Spear, Introduction: How Research on Alcohol and Alcoholism Has Informed Research on Developing Brains, in: J. H. Hannigan (Eds.), Alcohol and alcoholism : effects on brain and development, Lawrence Erlbaum Associates, London, 1999.
[9] J. H. Hannigan, R. F. Berman, Amelioration of fetal alcohol-related neurodevelopmental disorders in rats: exploring pharmacological and environmental treatments, Neurotoxicol. Teratol., 2000, 22:103-11.
[10] E. Ozer, S. Sarioglu, A. Gure, Effects of prenatal ethanol exposure on neuronal migration, neuronogenesis and brain myelination in the mice brain, Clin. Neuropathol., 2000, 19:21-5.
[11] G. I. Henderson, J. J. Chen, and S. Schenker. Ethanol, oxidative stress, reactive aldehydes, and the fetus, Front. Biosci., 1999, 4:D541-D550.
[12] R. J. Ward, Y. Zhang, R. R. Crichton, B. Piret, J. Piette, P. de Witte, Identification of the nuclear transcription factor NFκB in rat after in vivo ethanol administration, FEBS Lett., 1996, 389:119-22.
[13] N. Nakatsuji, Craniofacial malformation in Xenopus laevis tadpoles caused by the exposure of early embryos to ethanol, Teratology, 1983, 28:299-305.
[14] J. W. Olney, T. Tenkova, K. Dikranian, Y. Q. Qin, J. Labruyere, C. Ikonomidou, Ethanol-induced apoptotic neurodegeneration in the developing C57BL/6 mouse brain, Brain Res. Dev. Brain Res., 2002, 133:115-26.
[15] R. Natsuki, Effect of ethanol on brain biogenic amines and phospholipids in the chick embryo, Arukoru Kenkyuto Yakubutsu Ison, 1990, 25:497-508.
[16] M. J. Cano, A. Ayala, M. L. Murillo, O. Carreras, Protective effect of folic acid against oxidative stress produced in 21-day postpartum rats by maternal-ethanol chronic consumption during pregnancy and lactation period, Free Radic. Res., 2001, 34:1-8.

[17] J. J. Mitchell, M. Paiva, M. B. Heaton, The antioxidants vitamin E and beta-carotene protect against ethanol-induced neurotoxicity in embryonic rat hippocampal cultures, Alcohol, 1999, 17:163-8.
[18] B. M. Altura, A. Gebrewold, A. Zhang, B. T. Altura, Ethanol induces rapid lipid peroxidation and activation of nuclear factor-κB in cerebral vascular smooth muscle: relation to alcohol-induced brain injury in rats, Neurosci. Lett., 2002, 325:95-8.
[19] M. B. Toledano, W. J. Leonard, Modulation of transcription factor NF-kappa B binding activity by oxidation-reduction in vitro, Proc. Natl. Acad. Sci. U.S.A, 1991, 88:4328-32.
[20] Q. Dong, S. Kelkar, Y. Xiao, S. Joshi-Barve, C. J. McClain, S. S. Barve, Ethanol enhances TNF-alpha-inducible NFκB activation and HIV-1-LTR transcription in CD4+ Jurkat T lymphocytes, J. Lab Clin. Med., 2000, 136:333-43.
[21] P. Mandrekar, D. Catalano, G. Szabo, Alcohol-induced regulation of nuclear regulatory factor-kappa beta in human monocytes, Alcohol Clin. Exp. Res., 1997, 21:988-94.
[22] P. Mandrekar, D. Catalano, G. Szabo, Inhibition of lipopolysaccharide-mediated NFκB activation by ethanol in human monocytes, Int. Immunol., 1999, 11:1781-90.
[23] Z. Yao, J. Zhang, J. Dai, E. T. Keller, Ethanol activates NFκB DNA binding and p561ck protein tyrosine kinase in human osteoblast-like cells, Bone, 2001, 28:167-73.
[24] C. Guerri, C. Montoliu, J. Renau-Piqueras, Involvement of free radical mechanism in the toxic effects of alcohol: implications for fetal alcohol syndrome, Adv. Exp. Med. Biol., 1993, 366:291-305.
[25] J. R. West, Recent findings on the mechanisms by which alcohol damages the developing nervous system, Alcohol, 1994, Suppl. 2:395-399.
[26] G. I. Henderson, B. G. Devi, A. Perez, S. Schenker, In utero ethanol exposure elicits oxidative stress in the rat fetus, Alcohol Clin. Exp. Res., 1995, 19:714-720.
[27] S. L. Marklund, N. G. Westman, E. Lundgren, and G. Roos, Copper- and zinc-containing superoxide dismutase, manganese-containing superoxide dismutase, catalase, and glutathione peroxidase in normal and neoplastic human cell lines and normal human tissues, Cancer Res., 1982, 42:1955-1961.
[28] R. A. Floyd and J. M. Carney, Free radical damage to protein and DNA: mechanisms involved and relevant observations on brain undergoing oxidative stress, Ann. Neurol., 1992, 32:S22-S27.
[29] A. Aspberg and O. Tottmar, Ethanol-induced increase in catalase activity in reaggregation cultures of rat brain cells is due to increased oligodendrocyte differentiation, Alcohol.: Clin. Exp. Res., 1994, 18:620-624.
[30] H. Mover and A. Ar, Antioxidant enzymatic activity in embryos and placenta of rats chronically exposed to hypoxia and hyperoxia, Comp. Biochem. Physiol., Part C: Pharmacol., Toxicol. Endocrinol., 1997, 117:151-157.
[31] P. Walicke, S. Varon, and M. Manthrope. Purification of a human red blood cell protein supporting the survival of cultured CNS neurons, and its identification as catalase, J. Neurosci., 1986, 6:1114-1121.
[32] E. M. Martin, S. D. Skaper and S. Varon, Catalase protection of neuronal survival in vitro is not directed to the accumulation of peroxides in the culture medium, Int. J. Dev. Neurosci., 1987, 5:1-10.
[33] R. M. Chau, S. D. Skaper and S. Varon, Peroxidative block of glucose utilization and survival in CNS neuronal cultures, Neurochem. Res., 1988, 13: 611-616
[34] P. Callaerts, G. Halder and W. J. Gehring, PAX-6 in development and evolution, Annu. Rev. Neurosci, 1997, 20: 483-532.
[35] N. Osumi, The role of Pax6 in brain patterning, Tohoku J. Exp. Med., 2001, 193:163-174.
[36] N. Heins, P. Malatesta, F. Cecconi, M. Nakafuku, K. L. Tucker, M. A. Hack, P. Chapouton, Y. A. Barde and M. Gotz, Glial cells generate neurons: the role of the transcription factor Pax6, Nat. Neurosci., 2002, 5:308-315.
[37] A. Stoykova, R. Fritsch, C. Walther and P. Gruss, Forebrain patterning defects in small eye mutant mice, Development, 1996, 122:3453-3465.
[38] T. Pratt, T. Vitalis, N. Warren, J. M. Edgar, J. O. Mason and D. J. Price, A role for Pax6 in the normal development of dorsal thalamus and its cortical connections,Development, 2000, 127:5167-5178.
[39] Y. Peng, R. H. Xu, J. M. Mei, X. P. Li, D. Yan, H. F. Kung and J. M. Phang, Neural inhibition by C-jun as a synergizing factor in bone morphogenetic protein 4 signaling, Neuroscience, 2002, 109:657-664.
[40] K. Stratton, C. Howe, and F. Battaglia, (Eds), Fetal alcohol syndrome: diagnosis, epidemiology, prevention, and treatment, National Academy Press, Washington, D.C., 1996.
[41] C. Ikonomidou, P. Bittigau, M. J. Ishimaru, D. F. Wozniak, C. Koch, K. Genz, M. T. Price, V. Stefovska, F. Horster, T. Tenkova, K. Dikranian, and J. W. Olney, Ethanol-induced apoptotic neurodegeneration and fetal alcohol syndrome, Science, 2000, 287:1056-60.
[42] C. Guerri and J. Renau-Piqueras, Alcohol, astroglia, and brain development, Mol. Neurobiol., 1997, 15:65-81.
[43] M. Guizzetti and L. G. Costa, Possible role of protein kinase C zeta in muscarinic receptor-induced proliferation of astrocytoma cells, Biochem. Pharmacol., 2000, 60:1457-66.

[44] L. G. Costa and M. Guizzetti, Muscarinic cholinergic receptor signal transduction as a potential target for the developmental neurotoxicity of ethanol, Biochem. Pharmacol., 1999, 57:721-6.
[45] M. J. Eckardt, S. E. File, G. L. Gessa, K. A. Grant, C. Guerri, P. L. Hoffman, H. Kalant, G. F. Koob, T. K. Li, and B. Tabakoff, Effects of moderate alcohol consumption on the central nervous system, Alcohol Clin. Exp. Res., 1998, 22:998-1040.
[46] H. C. Becker, J. L. Diaz-Granados, and C. L. Randall, Teratogenic actions of ethanol in the mouse: a minireview, Pharmacol. Biochem. Behav., 1996, 55:501-13.
[47] C. E. Chmielewski, L. M. Hernandez, A. Quesada, J. A. Pozas, L. Picabea, and F. A. Prada, Effects of ethanol on the inner layers of chick retina during development, Alcohol, 1997, 14:313-7.
[48] B. Bupp, Sr. and I. A. Shibley, Jr., Teratogenicity of ethanol in different chicken strains, Alcohol, 1998, 33:457-64.

We claim:

1. A Xenopus laevis embryo model for Fetal Alcohol Syndrome (FAS), comprising:
a Xenopus laevis embryo that exhibits birth defects associated with Fetal Alcohol Syndrome (FAS), resulting from a specific condition of transient alcohol exposure of the embryo.

2. The model according to claim 1, wherein the specific condition includes exposure to at least about 0.4% to about 1% alcohol.

3. The model according to claim 2, wherein the exposure occurs between an initial neural plate stage and a late-neurula stage of growth of the embryo.

4. The model according to claim 2, wherein the exposure ranges from about 6 to about 12 hours.

5. The model according to claim 1, wherein the birth defects include edema, bended body axis, microcephaly, gut defects, eye anomalies, and growth retardation, and the specific condition includes exposure to about 0.4% to about 1% alcohol between an initial neural plate stage and a late neural stage of embryonic growth.

6. A method for screening an agent which is protective or therapeutic for FAS, comprising:
(i) administering the agent to the Xenopus laevis embryo model of claim 1 before, after or concurrently with the transient alcohol exposure of the embryo; and
(ii) detecting a reduction in birth defects in the embryo compared to a control.

7. The method of claim 6, wherein the reduction in birth defects is detected by observing a morphology of the embryo compared to a control.

8. The method of claim 6, wherein the reduction in birth defects is detected by measuring a level of neural marker expression in the embryo.

9. The method of claim 8, wherein the neural marker is Pax6, Otx2, Sox2, Sox3, and/or NCAM.

10. A method for obtaining a Xenopus embryo model of fetal alcohol syndrome, comprising:
subjecting the Xenopus embryo to a specific condition of transient alcohol exposure during embryonic development to produce tadpoles exhibiting birth defects consistent with FAS.

11. The method according to claim 10, wherein the specific condition includes an exposure to alcohol ranging from about 0.4% to about 1% alcohol.

12. The method according to claim 11, wherein the exposure occurs between an initial neural plate stage and a late-neurula stage of growth of the embryo.

13. The method according to claim 11, wherein the exposure occurs from about 6 to about 12 hours.

14. The method according to claim 10, wherein the birth defects include edema, bended body axis, microcephaly, gut defects, eye anomalies, and growth retardation.

15. A method of preventing or treating FAS comprising:
administering vitamin C to an animal at an amount effective to mitigate the effects of FAS.

16. The method according to claim 15, wherein the vitamin C is administered prior to an exposure of the animal to alcohol.

17. The method according to claim 15 wherein the vitamin C is administered concurrently with an exposure of the animal to alcohol.

18. A method of treating FAS comprising:
administering an amount of an agent effective to cause or enhance an expression of Pax6.

19. The method of claim 18, wherein the agent is vitamin C.

20. The method of claim 18, wherein the agent is a catalase.

21. A method of treating Fetal Alcohol Syndrome (FAS) comprising: administering an amount of an agent effective to cause suppression of NF-κB activation.

22. The method of claim 21, wherein the agent is vitamin C.

23. A method for preparing a Xenopus laevis embryo model for Fetal Alcohol Syndrome (FAS), comprising:
providing a Xenopus laevis embryo;
exposing the embryo to alcohol in an amount and for a time sufficient to induce birth defects characteristic of or associated with FAS.

24. A method in accordance with claim 22, wherein the embryo is exposed to alcohol in a concentration ranging from at least about 0.4% to about 1.0% alcohol, the time is from about 6 to about 12 hours, and the exposure occurs between an initial plate stage and a late-neurula stage of embryological development, and the birth defects include endema, bended body axis, microcephaly, gut defects, eye anomalies, and growth retardation.

25. An Xenopus laevis FAS model having birth defects characteristic of FAS created in accordance with the method of claim 23.

26. An Xenopus laevis FAS model having birth defects characteristic of FAS created in accordance with the method of claim 24.
